(19) United States
(12) Patent Application Publication
(10) Pub. No.: US 2014/0288594 A1
(43) Pub. Date: Sep. 25, 2014

(54) Dual Diameter, Dual Density ePTFE Suture

(71) Applicant: Genesee Biomedical, Inc., Denver, CO (US)

(72) Inventors: Hans-Joachim Shaefers, Homurg /Saar (DE); Robyn Peterson, Plymouth, MN (US); Woodrow G. Mathison, Centennial, CO (US)

(73) Assignee: Genesee Biomedical, Inc., Denver, CO (US)

(21) Appl. No.: 14/161,964

(22) Filed: Jan. 23, 2014

Related U.S. Application Data
(60) Provisional application No. 61/756,520, filed on Jan. 25, 2013.

(51) Int. Cl. A61B 17/06

(52) U.S. Cl. CPC A61B 17/06166; A61B 2017/06057
USPC 606/222; 606/228

(57) ABSTRACT

A suture for use in cardiac valve repair comprises a flexible rod of ePTFE (expanded polytetrafluoroethylene) formed with end sections of a first diameter and a center section of a second diameter, the second diameter being greater than the first diameter, and the end sections having a length sufficient to enable one to be fastened to the other. In one embodiment, the first diameter is about 1-1.6 mm and the second diameter is between about 1.8-4 mm. The center section can be about 2-9 cm in length. Surgical needles are preferably provided attached to the distal ends of the end sections. The center section has a density less than the end sections, the density of the center section being chosen to promote tissue ingrowth following implantation in heart tissue. Representative density range for the center section is 0.25-0.35 g/cm³.

### RELATED APPLICATIONS

This application claims priority from U.S. Provisional Patent Application Ser. No. 61/756,520, filed Jan. 25, 2013, entitled “Dual Diameter, Dual Density ePTFE Suture,” which is hereby incorporated by reference.

### TECHNICAL FIELD

The present invention is directed to surgical sutures, and more particularly to a dual diameter, dual density ePTFE suture for use in stabilizing the diameter of an aorto-ventricular junction of an aortic root.

### BACKGROUND

Aortic valve repair has evolved from an occasional procedure to a reproducible treatment option for many patients with aortic valve regurgitation over the last fifteen years. Aortic annuloplasty has shown increasing promise. Recently, the focus has been on remodeling and stabilizing the diameter of the aorto-extraventricular junction.

Different concepts of an annuloplasty approach have been proposed, including use of an external ring, internal ring and suture annuloplasty. Lansac, et al., have published on the use of an external aortic ring (Lansac, et al. (2010) J. Thorac. Cardiovasc. Surg. 140:528-535, “An aortic ring from physiological reconstruction of the root to a standardized approach of aortic valve repair”). However, because of anatomical variations between patients, the external ring has been found to not be usable in up to 30% of patients. Recently, Lansac, et al. have published information about an external open ring for use in aortic valve repair. They propose a prosthetic ring of an open configuration to allow its placement externally to the aorta and below the coronary arteries without detaching them from the aortic wall (Lansac, et al. (2011) Multimedia Manual of Cardiothoracic Surgery, doi:10.1510/mmcts.2009.004119, pp. 1-7, “An external open ring for isolated aortic valve repair”); see also Asano, at p. 2 (Asano, et al. (2012) Eur J Cardiothorac Surg Apr. 14, 2012 pp. 1-6, doi:10.1093/ejcts/ers120, “Mid-term results after sinutubular junction remodeling with aortic cusp repair”). While a potential improvement over a closed ring, the open ring is still relatively cumbersome to implant.

Also proposed in the literature has been the placement of a subannular PTFE suture tied around a Hegar dilator. (Kunihara, et al. (2012) J Thorac Cardiovasc Surg 143(6):1389-1395, “Preoperative aortic root geometry and postoperative cusp configuration primarily determine long-term outcome after valve-preserving aortic root repair”). While this technique holds promise, concerns exist about secure placement of the suture to avoid interference with the right and left coronary arteries. A modification of the described technique to implant a known PTFE suture in sub-right ventricular tissue to help stabilize the suture's position is one potential option, but this may create a possibility of erosion of the sub-right ventricular tissue contacted by the implanted known PTFE suture.

The various embodiments discussed below are directed toward overcoming one or more of the problems discussed above.

### SUMMARY OF THE EMBODIMENTS

The first aspect is a suture for use in cardiac valve repair. The suture comprises a flexible rod of ePTFE (expanded polytetrafluoroethylene) formed with end sections of a first diameter and a center section of a second diameter, the second diameter being greater than the first diameter, and the end sections having a length sufficient to enable one to be attached to the other. In one embodiment, the first diameter is between about 1-1.6 mm and the second diameter is between about 1.8-4 mm. The center section can be about 2-9 cm in length and each end section can be about 7-14 cm in length. Surgical needles may provided attached to the distal ends of the end sections. The center section has a density less than the end sections, the density of the center section being chosen to promote tissue ingrowth following implantation in heart tissue. Representative density range for the center section is 0.25-0.35 g/cm³.

A second aspect is a suture for use in circumclusion of a basal ring of an aortic root. The suture comprises a flexible rod of ePTFE formed with end sections of a first diameter and a center section of a second diameter. The center section has a length about equal to the length of right ventricular tissue adjacent to an outer circumference of the basal ring. The center section has a second diameter sufficient to minimize tissue abrasion following implantation in adjacent sub-right ventricular tissue. Further, the center section may have a density less than the end section density, the center section density promoting tissue ingrowth following implantation in sub-right ventricular tissue. The density range of the center section can be 0.25-0.35 g/cm³. The center section has a length of about 7 cm.

Another aspect of the invention is a method for stabilizing the diameter of an aorto-ventricular junction of an aortic root. The method comprises providing a suture made of ePTFE formed with end sections of a first diameter and a center section of a second diameter, the center section having a length about equal to the length of right ventricle tissue adjacent to an outer circumference of the basal ring of the aortic root, and the center section having a second diameter sufficient to minimize tissue abrasion following implantation in sub-ventricular tissue. A needle is attached to a distal end of each of the end sections. Leading with the needle, the suture is implanted in sub-right ventricular tissue adjacent to an outer circumference of the basal ring of the aortic root with the center section within the sub-right ventricular tissue. The aorto-ventricular junction is then formed to a select diameter by encircling the basal ring with the end sections and drawing the end sections together at the basal ring and then fastening the end sections to one another to fix the select diameter. The method may further include exiting the sub-right ventricular tissue before the membranous septum and below the right coronary artery, wherein the suture encircling the basal ring lies below the right coronary artery. The method may further include entering the needle into an outer wall of the aorta tangentially proximate the non-coronary sinus to attach an end section of the suture to the aorta at the basal ring. The method may further include attaching the suture to the aorta while encircling the basal ring using tacking sutures. The step of forming the aortic valve junction to a select diameter may include using an obturator.

### BRIEF DESCRIPTION OF THE DRAWINGS

FIG. 1 is a schematic representation of a dual diameter, dual density ePTFE suture;

FIG. 2 is a schematic representation of an aortic root of a human heart;

FIG. 3 is a schematic representation of a human heart with the atria removed;

FIG. 4 is a schematic representation of the heart of FIG. 3 with a dual diameter, dual density ePTFE suture partially installed around the basal ring of the aortic root; and

FIG. 5 is a schematic representation of the heart of FIG. 3 with a dual diameter, dual density ePTFE suture fully installed around the basal ring of the aortic root.

### DETAILED DESCRIPTION

Unless otherwise indicated, all numbers expressing quantities of ingredients, dimensions reaction conditions and so forth used in the specification and claims are to be understood as being modified in all instances by the term “about”.

In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

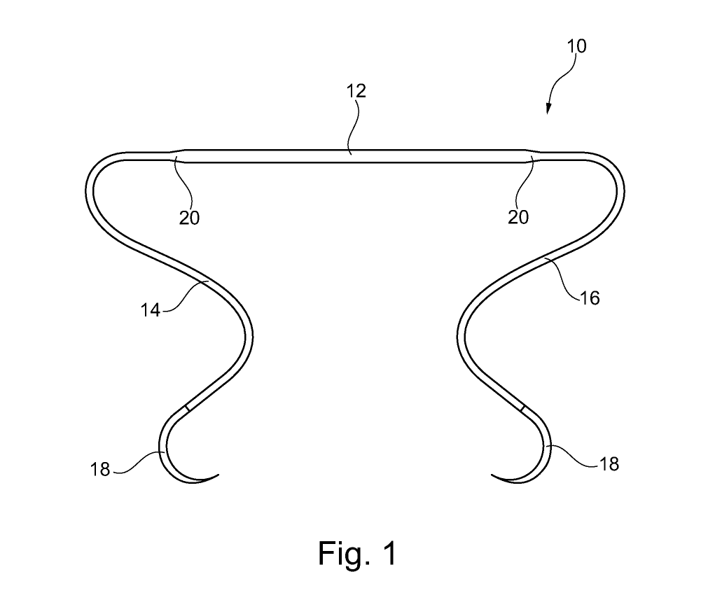

An embodiment of a dual diameter, dual density ePTFE suture 10 is illustrated in FIG. 1. As used herein, “ePTFE” means “expanded Polytetrafluoroethylene”, sometimes also referred to as “porous Polytetrafluoroethylene”. The dual diameter, dual density ePTFE suture 10 is a flexible rod of ePTFE having a center section 12 and end sections 14, 16 extending from each end of the center section 12. A needle 18 is provided at the distal end of each of the end sections 14, 16. The needles 18 may be attached to the distal ends of the end sections 14, 16 in any known manner, including crimping to the distal ends, using adhesives, or the like. Embodiments could also include a needle attached to only one distal end of the end sections 14, 16. The end sections 14, 16 are of a first diameter and the center section 12 is of a second diameter greater than the first diameter. While the embodiments illustrated herein show a cylindrical flexible rod, other cross-sectional configurations other than circular (such as various polygons) are within the scope of the invention. Use of “diameter” should therefore be understood to contemplate cross-sections other than circular and convey the idea of relative cross-sectional area. A taper 20 transitions between each end of the center section 12 and the end sections 14, 16. In one embodiment, center section 12, the end sections 14, 16 and the taper 20 are each integrally formed in a manufacturing process described in greater detail below.

In one alternative embodiment, the center section 12 could be a separate conduit of ePTFE received over a rod of ePTFE comprising the end sections 14, 16 and secured in place by an adhesive or other known means.

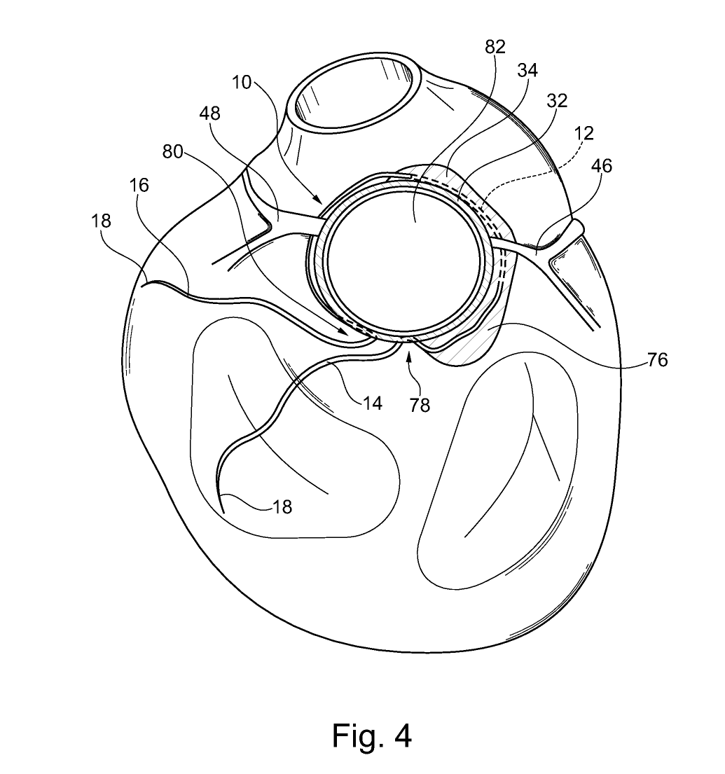

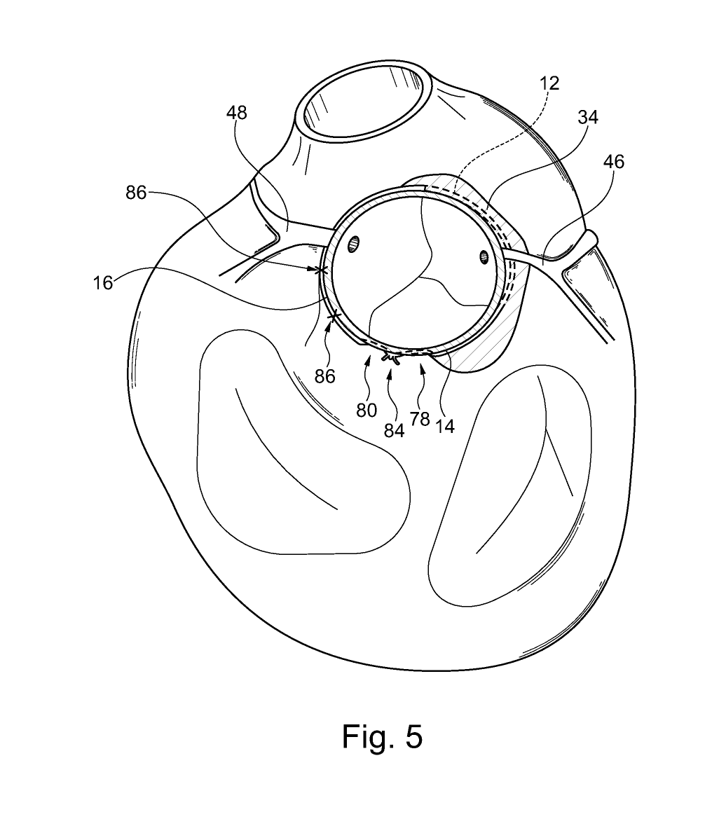

In the embodiment of FIG. 1, the center section 12 has a density which is less than the density of the end sections 14, 16. In various embodiments the center section 12 has a density promoting ingrowth of tissue when the dual diameter, dual density ePTFE suture 10 is implanted within human tissue. For example, when the center section 12 is implanted with the center section 12 residing in sub-right ventricular tissue, as will be described in greater detail below with reference to FIGS. 4 and 5.

The center section 12 has a length selected to be at least long enough to extend the length of sub-right ventricular tissue adjacent to an outer circumference of the basal ring of an aortic root, as will be discussed in greater detail below. In some embodiments the length may be selected to reside substantially entirely within sub-right ventricular tissue adjacent to an outer circumference of the basal ring of an aortic root. In one embodiment, the center section 12 has a length of about 7 cm. Because the length of the sub-right ventricular tissue adjacent to an outer circumference of the basal ring of an aortic root can vary from patient to patient, embodiments may include center sections having a length of between 2-9 cm, and even less than 2 cm or greater than 9 cm.

The end sections 14, 16 may be of a length shorter, equal to or greater than the length of the center section 12. In the embodiment illustrated in FIG. 1, the end sections 14, 16 are about equal in length to the center section 12.

The tapers 20 in various embodiments may have a length of between 2-5 mm. In other embodiments the length of the taper is less than 3 mm.

Embodiments may include end sections 14, 16 having a first diameter in a range of about 1-1.6 mm and the second diameter in a range of about 1.8-4 mm. Embodiments may also include the first diameter in a range of about 1.0-1.5 mm and a second diameter in a range of about 1.9-2.5 mm. Embodiments may also include the first diameter being in a range of about 1.3-1.5 mm and the second diameter being in the range of about 1.9-2.1 mm.

Embodiments may include the end sections 14, 16 having a density in a range of about 0.4-0.7 grams per cubic cm (g/cm³) and the center section 12 having a density in a range of about 0.25-0.35 g/cm³. Other embodiments may include the end sections 14, 16 having a density in a range of about 0.5-0.65 g/cm³ and the center section 12 having a density in a range of about 0.25-0.35 g/cm³.

The embodiment illustrated in FIG. 1 shows end sections 14, 16 having a first diameter a center section 12 having a second diameter. Other embodiments could include one or more additional linear sections having a diameter different than at least one of the first or second diameter. For example, a separate segment from the center section 12 could be provided spaced lengthwise from the center section 12 along one of the end sections 14, 16 for a select length and have a diameter greater than the first diameter, such as equal to the center section 12 Likewise this additional section of increased diameter from the end sections 14, 16 could have a density in the ranges of the center section 12 discussed above.

In one particular embodiment, a suture is provided for use in circumclusion of a basal ring of an aortic root. The suture may have the structure as described above with respect to FIG. 1. The suture comprises a flexible rod of dual diameter, dual density ePTFE 10 formed with end sections 14, 16 of a first diameter and a center section 12 of a second diameter. The center section has a length about equal to or greater than the length of right ventricle tissue adjacent to an outer circumference of the basal ring. The center section 12 further has a second diameter sufficient to minimize tissue abrasion following implantation in adjacent sub-right ventricular tissue. The center section 12 has a density less than a density of the end sections, the center section 12 density promoting tissue ingrowth following installation in heart tissue, for example, sub-right ventricular tissue.

One particular embodiment of a dual diameter, dual density ePTFE suture 10 provides end sections 14, 16 of a first diameter of about 1.37 mm and a density of 0.65 g/cm³ and a center section 12 having a second diameter of about 2.0 mm and a density of about 0.31 g/cm³. Another particular embodiment has end sections 14, 16 of a first diameter of about 1.5 mm and a density of about 0.52 g/cm³ and a center section 12 having a diameter of about 2.0 mm and a density of about 0.3 g/cm³.

The ePTFE suture as described above is manufactured by an extrusion process. In a controlled environment for temperature a supply of sifted fine powder PTFE resin is combined with a high grade Hydrocarbon Lubricant which is typically a selected grade of Isopar solvents. The PTFE resin and the Hydrocarbon lubricant is then blended to ensure even coverage of the PTFE resin with the lubricant. The combination of the resin and lubricant is also referred to as “paste”. Under low pressures, the paste is compressed in a cylindrical tube. The compressed paste is processed to remove air volumes from the mixture, increase the amount of resin that can be extruded, and to provide an easily handled form of the paste to be loaded into a paste or ram extruder. The preformed resin in a form is also called a “billet”.

The preformed resin or billet is loaded into a vertically or horizontally orientated ram extruder. A ram extruder is a pneumatic or hydraulic press comprised of a barrel, die, mandrel, and ram plate. Under high pressure, the ram plate forces the billet thru the extrusion barrel and past the die and mandrel to create a predetermined geometric shape, in the case of the embodiment of FIG. 1, a cylindrical shape. The shear forces generated by the paste being forced thru the die and mandrel not only shape the paste but cause the PTFE resin to fibrillate. Fibrillation is the formation and orientation of PTFE resin particles to long chains or fibers that run down the length of an extruded profile. Depending on resin and extrusion requirements, low temperature heating of extrusion tooling is sometimes used to assist ram extrusion of PTFE paste. Each extruded profile is cut to a specific length that is dependent on the final volume density or porosity required.

Following extrusion is tying. Tying involves the crimping or tying of plugs or rings to both ends of a cut and extruded profile to assist in the handling and secondary processing of each tube. The mechanical fixtures and methods used are specific to the shape and secondary processing required by the product to be manufactured.

The next step is drying to remove the hydrocarbon lubricant. Drying applications occur in well ventilated processing areas with the optional use of heat to assist in the speed and removal of lubricant. The lubricant is used to only assist the extrusion and fibrillation of the PTFE resin.

Tied and dried extruded profiles are loaded into high temperature expansion mechanisms between the temperatures of 200° C. and 300° C. The extruded profiles are stretched in the extrusion direction by the plugs or ties secured to the ends of each extruded profile. The amount of stretch or elongation that each extruded profile incurs is dependent on the final density or porosity required for the final application. The expansion processes creates a complex matrix of a large number of nodes and interconnected by fibers. The long axis of the nodes is perpendicular to the direction of the stretch with long thin fibers running between each node. The greater the amount of stretch, the longer the length of the fibers will be and the narrower that the nodes will appear.

Unexpanded PTFE tubing has a volume density of 2.15 g/cm³ while expanded ePTFE tubing can have a volume density range between 0.2-1.4 g/cm³ which correlates to an air volume between 90-35%. Porosity of tubing is an inverse relationship to density.

The expanded profile is removed from its mechanical fixtures that were used in the tying processes and used in the drying and expansion operations. The ends of the expanded profile are then passed thru a conical compression die that exhibits the general final diameters needed for the end sections. The radial reduction of the expanded profile reduces the diameter and also increases the density in the die reduced areas of the profile. Radial reduction tooling may or may not be heated to assist in the radial compression. Once the reduction of the profile is completed the processed units are then re-attached to its mechanical fixtures to facilitate the final operation of sintering.

Expanded PTFE profiles are retained in tension by the plugs or ties and then placed in a high temperature oven exceeding 320° C. The exposure of expanded PTFE material alters the physical properties from amorphous to a more stable crystalline state. The temperature and duration that expanded profiles are sintered is dependent on porosity and mass of the extruded profile. It is not until the extruded profile is exposed to significant amounts of elevated heat that the final profile is able to be handled without significant and permanent damage to the profile geometry. Thereafter the needles can be added to the distal ends of the end sections by known techniques.

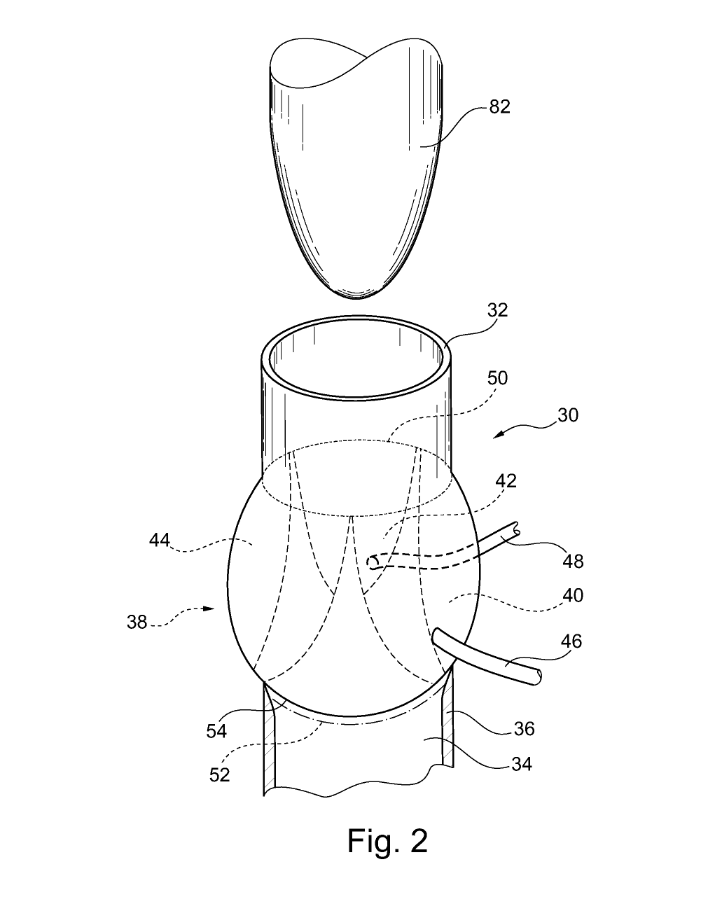

FIG. 2 is a schematic representation of an aortic root 30 for the purpose of illustrating the use of the various embodiments dual diameter, dual density ePTFE suture 10 described above. The aortic root 30 comprises a portion of an aorta 32 extending from the right ventricle 34 with sub-right ventricular tissue 36 shown in cross-section. The aortic root 30 also includes the aortic valve 38 shown in broken lines to indicate its position within the aortic root. The aortic valve 38 comprises a right coronary leaflet 40, a left coronary leaflet 42 and a non-coronary leaflet 44. The right coronary artery 46 is shown proximate the right coronary leaflet 40 and the left coronary artery 48 is shown proximate the left coronary leaflet 42. The leaflet tops are attached at the Sino tubular junction 50 and the bottoms of the leaflets extend to a basal ring, sometimes called a virtual basal ring 52 which lies below an aorto-ventricular junction 54. In use, and as will be described in greater detail below, the dual diameter, dual density ePTFE suture 10 is implanted around the virtual basal ring 52 in substantially a plane defined by the virtual basal ring 52.

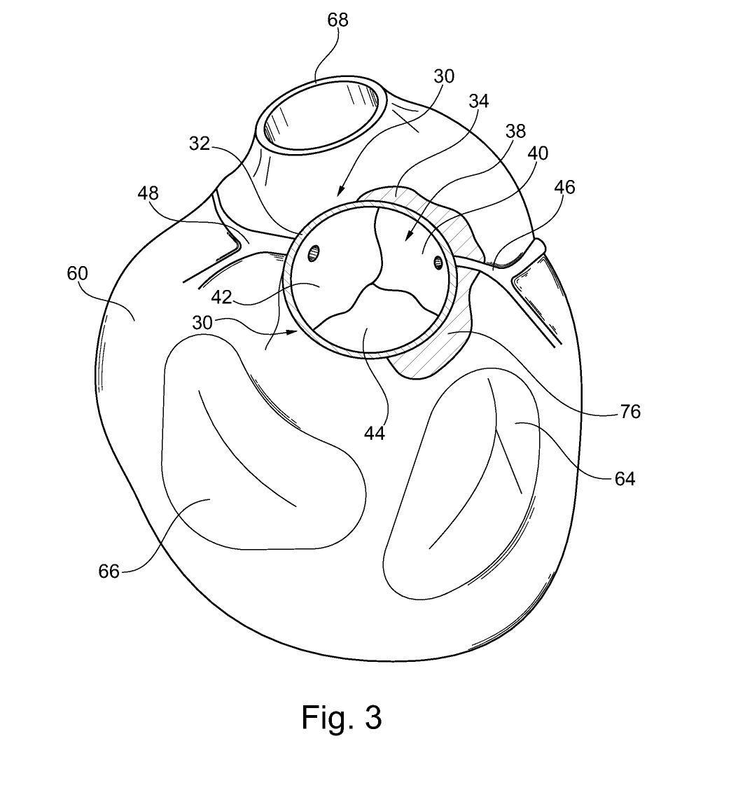

FIG. 3 is a schematic representation of a human heart 60 with the atria removed. FIG. 3 shows the aortic valve 38 and a cross-section of a segment of the aorta 32 comprising the aortic root 30 surrounded by the tricuspid valve 64, the mitral valve 66 and a segment of the pulmonary artery 68. Also visible is the right coronary leaflet 40, left coronary leaflet 42 and the non-coronary leaflet 44. The right coronary artery 46 is shown extending from the aorta in the proximity of the right coronary leaflet 40 and the left coronary artery 48 is shown extending from the aorta in the proximity of the left coronary leaflet 42. Also shown schematically is a segment or length of the right ventricle 34 adjacent to an outer circumference of the basal ring of the aortic root 30 (see also FIG. 2). The area of the membranous septum 76 is also illustrated.

FIG. 4 illustrates one embodiment of a method of implanting the dual diameter, dual density ePTFE suture 10 around an aortic root 30 for the purpose of stabilizing the diameter of the aorto-ventricular junction 54. Referring to FIG. 4, the aorta 32 is shown as severed for the purpose of illustration. In practice, the aorta is cut transverse its axis to allow access thereto, but is typically not required to be severed entirely unless perhaps other procedures directed to the aortic root are necessary. The dual diameter, dual density ePTFE suture 10 is implanted by inserting the needle 18 associated with the end section 14 clockwise as viewed in FIG. 4 into the sub-right ventricular tissue 36 (see FIG. 2) and passing the needle 18 through the sub-right ventricular tissue 36 of the right ventricle 34 beneath the right coronary artery 46. The needle exits the right ventricle 34 before encountering the area of the membranous septum 76 to avoid potential atrioventricular (“AV”) block. In the illustrated embodiment the needle 18 enters the outer wall of the aorta 32 tangentially proximate the non-coronary leaflet 44 (the coronary sinus) as indicated at 78. The end section 16 is positioned to encircle the aortic root opposite the direction of end section 14, or counter clockwise as depicted in FIG. 4, at the level of the basal ring 52 and is fed under the left coronary artery 48. The needle 18 of the end section 16 can be used to enter an outer wall of the aorta 32 proximate the non-coronary sinus to attach the end section 16 of the suture to the wall of the aorta 32 as indicated at 80. An obturator 82 placed within the aortic root 30 provides a select diameter of the aorto-ventricular junction 54. After course placement as indicated in FIG. 4, the end section 14 can be pulled further clockwise to completely draw the center section 12 of the dual diameter, dual density ePTFE suture 10 into the sub-right ventricular tissue 36. Thereafter the ends sections 14, 16 of the suture can be tensioned to conform the aorto-ventricular junction 54 diameter to that of the obturator 82 and the end sections 14, 16 can be tied into a knot 84 as illustrated in FIG. 5, or fastened together by other know means. Thereafter the obturator 82 is removed, as viewed in FIG. 5. In some embodiments of the method, the dual diameter, dual density ePTFE suture 10 can be attached to the aorta 32 using tacking sutures 86 to better retain the dual diameter, dual density ePTFE suture 10 about the virtual basal ring 52 of the aortic root 30. Thereafter the aorta 32 can be sutured to its original form and the surgery completed.

Variations of the embodiment of the method discussed above may be dictated by anatomical features of a particular patient, but in almost all applications the dual diameter, dual density ePTFE suture 10 and the method as described herein allow for circumclusion of a basal ring of an aortic root with a suture implanted proximally of both the right coronary artery 46 and the left coronary artery 48 substantially in the plane of the basal ring 52. The relatively large diameter of the center section 12 of the dual diameter, dual density ePTFE suture 10 minimizes or prevents abrasion of the sub-right ventricular tissue 36 or the membranous septum 76 once implanted therein. In addition, the relatively low density of the center section 12 enhances tissue ingrowth following implanting in the sub-right ventricular tissue 36 to further stabilize the implanting of the suture over time. The relatively less dense end sections 14, 16 of the suture are of a diameter that may facilitate attaching the suture to the aorta without fully penetrating entirely the wall of the aorta. In addition, the smaller diameter end sections 14, 16 of the suture may be readily tied together. In some embodiments the density of the end sections 14, 16 is low enough than the knot 84 compresses the suture material forming an extremely stable and secure knot. Embodiments could also include using some manner of attachment structure such as a clip or the like instead of the knot 84 illustrated in FIG. 5. Use of the step of attaching the suture to the aorta 32 by tangentially entering the exterior wall of the aorta 32 or by the use of tacking sutures 84, or both, is within the discretion of the surgeon as dictated by the patient's anatomy.

The use of the dual diameter, dual density ePTFE suture 10 for the circumclusion of the basal ring of the aortic root 30 eliminates concerns of crimping or occluding the right and left coronary arteries 46, 48 while providing a safe, stable implanted suture. The procedure can be conducted by an experienced surgeon in a relatively short period of time (e.g., less than 20 minutes), minimizing the time the patient must be on pump bypass, a significantly shorter period of time than implanting alternative devices for stabilization of the aorto-ventricular junction 54. In addition, it allows for stabilization at the level of the anatomical basal ring, even if the aortoventricular junction is located more cranially.

Various embodiments of the disclosure could also include permutations of the various elements recited in the claims as if each dependent claim was a multiple dependent claim incorporating the limitations of each of the preceding dependent claims as well as the independent claims. Such permutations are expressly within the scope of this disclosure.

The description of the present embodiments has been presented for purposes of illustration and description, but is not intended to be exhaustive or limiting of the invention to the form disclosed. The scope of the present invention is limited only by the scope of the following claims. Many modifications and variations will be apparent to those of ordinary skill in the art. The embodiment described and shown in the figures was chosen and described in order to best explain the principles of the invention, the practical application, and to enable others of ordinary skill in the art to understand the invention for various embodiments with various modifications as are suited to the particular use contemplated. All references cited herein are incorporated in their entirety by reference.

What is claimed is:

1. A suture for use in cardiac valve repair, the suture comprising a flexible rod of ePTFE formed with end sections of a first cross-sectional area and a center section of a second cross-sectional area, the second cross-sectional area being greater than the first cross-sectional area, and end sections having a length sufficient to enable one to be fastened to the other.

2. The suture of claim 1 wherein the end sections and the center section are integrally formed of ePTFE.

3. The suture of claim 1 further comprising a taper between each end section and the center section.

4. The suture of claim 1 wherein the center section is about 2-9 cm in length.

5. The suture of claim 4 wherein each end section is about 7-14 cm in length.

6. The suture of claim 1 wherein the first and second cross-sectional areas are circular and have a diameter, the first diameter being between about 1-1.6 mm and the second diameter being between about 1.8-4 mm.

7. The suture of claim 6 wherein the second diameter is between about 1.9-2.5 mm.

8. The suture of claim 7 further comprising a taper between each end section and the center section, the taper being less than 3 mm in length.

9. The suture of claim 1 wherein the end sections have a greater density than the center section.

10. The suture of claim 9 wherein the end sections have a density in a range of about 0.4-0.7 grams per cubic centimeter (g/cm³) and the center section has a density in a range of about 0.25-0.35 g/cm³.

11. The suture of claim 9 wherein the end sections have a density in a range of about 0.5-0.65 grams per cubic centimeter (g/cm³) and the center section has a density in a range of about 0.25-0.35 g/cm³.

12. The suture of claim 1 wherein the end sections have a diameter in a range of about 1.37-1.50 mm and a density in a range of about 0.52-0.65 grams per cubic centimeter (g/cm³) and the center section has a diameter of about 2.0 mm and a density in a range of about 0.30-0.31 g/cm³.

13. The suture of claim 1 further comprising one or more additional linear sections having a cross-sectional area different than at least one of the first or second cross-sectional areas.

14. A suture for use in a circumclusion of a basal ring of an aortic root , the suture comprising a flexible rod of ePTFE formed with end sections of a first diameter and a center section of a second diameter, the center section having a length about equal to the length of right ventricle tissue adjacent to an outer circumference of the basal ring, and the center section having a second diameter sufficient to minimize tissue abrasion following implantation in adjacent sub-right ventricular tissue.

15. The suture of claim 14 wherein center section has a center section density less than an end section density, the center section density promoting tissue ingrowth following implantation in sub-right ventricular tissue.

16. The suture of claim 15 wherein the center section has a density in a range of about 0.25-0.35 g/cm³.

17. The suture of claim 16 wherein the center section is about 7 cm in length.

18. The suture of claim 14 wherein the first diameter is a range of about 1-1.5 mm and the second diameter is about 2 mm.

19. A method for stabilizing the diameter of an aorto-ventricular junction of an aortic root, the method comprising:
providing a suture comprising ePTFE formed with end sections of a first diameter and a center section of a second diameter, the center section having a length greater than or about equal to the length of right ventricle tissue adjacent to an outer circumference of the basal ring of the aortic root, and the center section having a second diameter sufficient to minimize tissue abrasion following implantation in sub-ventricular tissue;
attaching a needle to a distal end of each of the end sections;
leading with the needle, implanting the suture in sub-right ventricular tissue adjacent to an outer circumference of a basal ring of the aortic root with the center section within the sub-right ventricular tissue;
forming the aorto-ventricular junction to a select diameter by encircling the basal ring with the end sections and drawing the end sections together at the basal ring; and
fastening the end sections to one another to fix the select diameter.

20. The method of claim 19 further comprising the needle exiting the sub-right ventricular tissue before the membraneous septum and below the right coronary artery, wherein with the suture encircling the basal ring lies below the right coronary artery.

21. The method of claim 20 further comprising entering the needle into an outer wall of the aorta tangentially proximate the non-coronary sinus to attach an end section of the suture to the aorta.

22. The method of claim 20 further comprising attaching the suture to the aorta while encircling the basal ring at the level of the basal ring using tacking sutures.

23. The method of claim 20 wherein the step of forming the aortic valve junction to a select diameter includes using an obturator.
